(19) United States
(12) Patent Application Publication
(10) Pub. No.: US 2010/0285053 A1
(43) Pub. Date: Nov. 11, 2010

(54) Immunosuppressive Extract Of Cordyceps Sinensis And Uses Thereof

(75) Inventors: Timothy Dominic Gerard Lee, Halifax (CA); Julie Lynn Jordan, Halifax (CA)

Correspondence Address: FENWICK & WEST LLP, SILICON VALLEY CENTER, 801 CALIFORNIA STREET, MOUNTAIN VIEW, CA, 94041, US

(73) Assignee: Dalhousie University, Halifax (CA)

(21) Appl. No.: 12/303,922

(22) PCT Filed: Jun. 11, 2007
(86) PCT No.: PCT/IB07/02634

§ 371 Date: Apr. 5, 2010

Related U.S. Application Data
(60) Provisional application No. 60/812,203, filed on Jun. 9, 2006.

(51) Int. Cl. A61K 36/068; A61P 43/00

(52) U.S. Cl. 424/195.15

(57) ABSTRACT

The present invention described herein relates to aqueous extracts of Cordyceps sinensis for use as an immunosuppressant agent.

This application claims the benefit of the U.S. Provisional application No. 60/812,203, filed on Jun. 9, 2006, which is herein incorporated by reference in its entirety.

### TECHNICAL FIELD OF THE INVENTION

The present invention relates to aqueous extracts of Cordyceps sinensis for use as an immunosuppressant agent.

### BACKGROUND

Immunosuppressive agents are used in a variety of treatments where it is necessary to suppress the activity of the immune system. They are used, for example, to prevent rejection of transplanted organs and tissues, and for other diseases, including autoimmune diseases. Many of the agents have numerous side effects. Furthermore, most agents currently being used or investigated are useful for acute rejection, but not chronic rejection. Accordingly, in view of the few choices for immunosuppressive agents with low toxicity profiles and manageable side effects, and which can also be used for chronic organ or tissue rejection, there exists a need for identifying alternative immunosuppressive agents.

Cordyceps sinensis is a parasitic fungus that infects specific Lepidoptera sp. larvae, eventually killing the caterpillar and sprouting a fruiting body from its remaining shell. It has been used in Traditional Chinese Medicine for treatment of a variety of diseases. (Yue, et al., in Advanced Study for Traditional Chinese Herbal Medicine, Vol. 1, Institute of Materia Medica, Beijing Medical University and China Peking Union Medical University Press: Beijing, 1995, 91-113.)

### SUMMARY

The invention provides methods for treating chronic rejection following organ, tissue or cell transplantation by administering to an animal an aqueous extract of Cordyceps sinensis. The invention also provides mixtures and pharmaceutical compositions comprising aqueous extracts of Cordyceps sinensis.

In a first aspect, the invention provides a method for preventing or treating chronic rejection following organ, tissue or cell transplantation, by administering to an animal an effective amount of an aqueous extract of Cordyceps sinensis. In an embodiment of the first aspect, the transplant organ, tissue or cells are selected from kidney, heart, heart valve, arteries, vessels, liver, lung, peripheral stem cells, pancreas, cornea, small bowel or skin.

In a second embodiment, the aqueous extract consists of molecules of 500, 300, 200, 100 or 50 kDa or less.

In a third embodiment, the aqueous extract of Cordyceps sinensis prevents, inhibits or reduces allograft vasculopathy and/or reduces neointimal formation in arteries or vessels.

In a fourth embodiment, the method further comprises administering an immunosuppressant. The immunosuppressant can be Cyclosporin A, FK506 or other calcineurin inhibitors, Rapamycin, MMF, Azathioprine, FTY720, Everolimus or kinase inhibitors. More than one immunosuppressant may be used in combination with ACE.

In a second aspect, the invention provides a mixture comprising an aqueous extract of Cordyceps sinensis consisting of molecules of 500, 300, 200, 100 or 50 kDa or less.

In a third aspect, the invention provides a method of preparing an aqueous extract of Cordyceps sinensis comprising molecules of 300 kDa or less or 100 kDa or less, wherein an aqueous extract of Cordyceps sinensis is passed through a filter.

In a fourth aspect, the invention provides a pharmaceutical composition that includes an aqueous extract of Cordyceps sinensis and a pharmaceutically acceptable carrier. In embodiments, the extract of Cordyceps sinensis comprises molecules of 300 kDa or less or 100 kDa or less. In another embodiment of the fourth aspect, the pharmaceutical composition further comprises an immunosuppressant. The immunosuppressant can include Cyclosporin A, FK506 or other calcineurin inhibitors, Rapamycin, MMF, Azathioprine, FTY720, Everolimus or kinase inhibitors.

Additional aspects and embodiments of the invention are described below.

### BRIEF DESCRIPTION OF THE DRAWINGS

FIG. 1 shows the effect of Aqueous Cordyceps extract (ACE) on IFN-γ in primed splenocytes and isolated T-cells.

FIG. 2 shows the effect of ACE on IL-6 production in macrophages.

FIG. 3 shows the effect of different molecular weight fractions of ACE on IL-6 by macrophages.

FIG. 4 shows the effect of ACE in conjunction with cyclosporine A (CyA) on acute rejection following cardiac transplantation in rats.

FIG. 5A is a graph of transplanted heart survival measured by palpation score. FIG. 5B is a Kaplan-Meier plot of the survival of transplanted hearts from mice given cyclosporine A, ACE or a combination.

FIG. 6 shows the effect of ACE plus Cyclosporin A on allograft vasculopathy in chronic rejection in an abdominal aortic transplant model in mice and rats.

FIG. 7 shows the effect of fractioned ACE consisting of molecules of less than 100 kDa on allograft vasculopathy in chronic rejection in an abdominal aortic transplant model in mice.

FIG. 8 shows the effect of ACE on gene expression in peritoneal elicited murine macrophages.

FIG. 9A shows the effect of ACE on TNF-α production by macrophages. FIG. 9B shows the effect of ACE +IFN-γ on IL-6 production by macrophages. FIG. 9C shows the effect of ACE on nitric oxide production by macrophages.

FIG. 10 shows the effect of ACE on cytokine production by macrophages from TLR4 deficient mice.

FIG. 11 shows the effect of MAPK inhibitors on ACE activity.

FIG. 12 shows the effect of ACE on the phosphorylation of MAPK proteins.

FIG. 13A shows the effect of polymyxin B on ACE and LPS. FIG. 13B shows the effect of heat on LPS and ACE. FIG. 13C compares the effect of LPS and ACE on gene expression in macrophages.

### DETAILED DESCRIPTION

### I. Definitions

As used herein, chronic rejection means late graft rejection, clinically appearing (in humans) one year post transplant or later. It occurs in all solid organ transplants and is due to allograft vasculopathy and other complications. For example, in hearts, chronic rejection is primarily due to allograft vasculopathy. In kidney, chronic rejection is primarily due to allograft vasculopathy, tubular damage and interstitial fibrosis. In lung, a condition called bronchiolitis obliterans with pathological similarities to graft vasculopathy is the primary manifestation of chronic rejection.

As used herein, acute rejection means acute cell rejection, which, if left untreated, would result in graft failure within weeks to months.

The abbreviation “ACE” means Aqueous Cordyceps sinensis Extract.

II. Cordyceps sinensis Extract

Cordyceps sinensis is a parasitic fungus that infects specific Lepidoptera sp. larvae, eventually killing the caterpillar and sprouting a fruiting body from its remaining shell. It has been used in Traditional Chinese Medicine for treatment of a variety of diseases. Recent preliminary evidence has become available to suggest that this fungus might have immunosuppressive activity.

The Cordyceps extract described herein is produced by an aqueous extraction procedure where dried cultured mycelia are homogenized in water, boiled, centrifuged to remove particulates, freeze-dried, reconstituted in water at a known concentration and sterilized by filter sterilization. Cordyceps extract can also be prepared using temperatures ranging from 4° C. to 100° C., 20° C. or higher, 30° C. or higher, 40° C. or higher, 50° C. or higher, 60° C. or higher, 70° C. or higher, 80° C. or higher, 90° C. or higher, or 95° C. or higher. The aqueous extract can also be prepared using steam pressure, such as in an autoclave at more than 100° C. at a pressure higher than atmospheric pressure (approximately 14.7 pounds per square inch (psi). For example, the extract can be prepared at a temperature between 110° C. and 132° C. at 15-30 psi.

This Aqueous Cordyceps sinensis extract, or ACE, can be further fractionated to separate molecules in the extract on the basis of size or other properties, using filters, sizing columns, such as gel filtration, ion exchange columns, affinity purification, HPLC, and other separation methods. Active ACE, comprising immunosuppressive properties, can thus consist of molecules of 500, 400, 350, 300, 250, 200, 150, 100, or 50 kDa or less.

The dried mycelia can also be extracted with other hydrophilic solvents, including, without limitation, ethanol, methanol, propanol, isopropanol and acetone. The mycelia can also be extracted with a combination of one or more hydrophilic solvents and one or more hydrophobic solvents. Hydrophobic solvents include, without limitation, hexane and chloroform.

### III. Uses of ACE

The ACE is useful in the treatment and/or prevention of diseases or disorders mediated by leukocyte interactions, for example in transplantation, such as acute or chronic rejection of cell, tissue or organ allo- or xenografts or delayed graft function, allograft vasculopathy, naturally occurring arteriosclerosis, restenosis, coronary angioplasty restenosis, any surgical bypass failure in peripheral artery disease, coronary artery disease, restenosis after carotid endarterectomy, any peripheral vascular stenting procedures, bronchiolitis obliterans, biliary response in hepatic/liver transplant, chronic renal rejection, graft versus host disease, autoimmune diseases, e.g. rheumatoid arthritis, systemic lupus erythematosus, Hashimoto's thyroiditis, multiple sclerosis, myasthenia gravis, diabetes type I and disorders associated therewith, vasculitis, Sjögren's syndrome, uveitis, psoriasis, Graves disease, alopecia areata and others, contact dermatitis, inflammatory diseases optionally with underlying aberrant reactions, e.g. inflammatory bowel disease, Crohn's disease or ulcerative colitis, inflammatory lung injury, inflammatory liver injury, inflammatory glomerular injury, atherosclerosis, osteoarthritis, irritant contact dermatitis and further eczematous dermatitises, seborrhoeic dermatitis, cutaneous manifestations of immunologically-mediated disorders, inflammatory eye disease, myocarditis, T cell lymphomas or T cell leukemias, or adult respiratory distress syndrome.

Examples of cell, tissue or solid organ transplants include, e.g., pancreatic islets, stem cells, including hemopoetic stem cells, corneal tissue, neuronal tissue, heart (Zhang, et al., J. Tongji Med Univ., 10:100-108, 1990), lung, combined heart-lung, kidney, liver, bowel, skin (Zhu and Yu, Zhong Xi Yi Jie He Za Zhi, [Journal of Modern Developments in Chinese Medicine], 10:485-7, 1990; Cheng, et al., Zhong Xi Yi Jie He Xue Bao [Chinese Journal of Integrative Medicine], 4:185-88, 2006), pancreas, trachea or oesophagus. For the above uses the required dosage will, of course, vary depending on the mode of administration, the particular condition to be treated and the effect desired.

Chronic rejection is characterized, in general, by vasculopathy and a progressive loss of organ function. Its pathogenesis probably involves both humoral and cellular immune mechanisms. Chronic rejection may be mediated by a low-grade, persistent delayed type hypersensitivity response in which activated macrophages secrete mesenchymal cell growth factors. Chronic rejection may also reflect chronic ischemia secondary to injury of blood vessels by antibody or cell-mediated mechanisms. Vascular occlusion may occur as a result of α-actin positive smooth muscle-like cell proliferation in the intima of arterial walls. Chronic rejection may be due to cell mediated damage to the medial layer of the artery wall, which causes a pathological tissue repair mechanism resulting in neointimal thickening and vessel occlusion.

Allograft vasculopathy of cardiac grafts consists of a concentric thickening of the intimal layer of the epicardial coronary arteries leading to luminal occlusion, thrombosis and eventually ischemic organ failure. In addition, allograft vasculopathy is characterized by the presence of an immune infiltrate composed of lymphocytes and mononuclear cells in the adventitia and the loss of smooth muscle cells from the medial layer. (See, e.g., Skaro, et al., Cardiovasc. Res., 65:283-91, 2005.) Current immunosuppressive regimens do not inhibit allograft vasculopathy and conventional revascularization strategies are ineffective because of the diffuse nature of the disease. (Hosenpud, et al., J. Heart Lung Transplant, 20:805-15, 2001.) Indeed, cyclosporin A at a dose that prevents cardiac rejection does not prevent vasculopathy in either cardiac or aortic transplants in mice (Vessie, et al., Transplant Immunol., 15: 35-44, 2005).

CD8⁺ T-cell activity may play a role in allograft vasculopathy. Evidence indicates that CD8⁺ T-cells contribute to vascular remodeling characteristic of allograft vasculopathy. Cylosporin A was effective in preventing allograft vasculopathy in mice lacking CD8⁺ T-cells (Vessie, et al., Transplant Immunol., 15: 35-44, 2005). In addition, with an immunosuppressant, such as cyclosporin A, CD4⁺ T-cell function is ablated, but CD8⁺ T-cell function remains partially intact, suggesting that non-CD8⁺ T-cell effector mechanisms are sensitive to calcineurin inhibitor therapy (such as cyclosporin A), but CD8⁺ T-cell mediated allograft vasculopathy is refractory to such treatment (Skaro, et al., Transplant Immunol., 14:27-35). Furthermore, adoptive transfer of primed wild type CD8⁺ T-lymphocytes into immunodeficient recipients of aortic allografts resulted in the development of robust allograft vasculopathy lesions, indicating that CD8⁺ T-cells mediate allograft vasculopathy (Skaro, et al., Cardiovasc. Res., 65:283-91, 2005).

Because Cordyceps sinensis extract, particularly the fraction containing molecules of less than 100 kDa, can be used to reduce or ablate allograft vasculopathy in transplanted organs, including hearts and vascular grafts, it may be acting to reduce or ablate the activity of CD8⁺ T-cells.

The ACE may be administered as the sole active ingredient or in conjunction with, e.g. as an adjuvant to, other drugs e.g. immunosuppressive or immunomodulating agents or other anti-inflammatory agents, e.g. for the treatment or prevention of allo- or xenograft acute or chronic rejection or inflammatory or autoimmune disorders, or a chemotherapeutic agent, e.g. a malignant cell anti-proliferative agent. For example the ACE may be used in combination with a calcineurin inhibitor, e.g. cyclosporin A or FK 506; an mTOR inhibitor, e.g. Rapamycin, 40-O-(2-hydroxyethyl)-rapamycin, CCI779, ABT578 or AP23573; an ascomycin having immunosuppressive properties, e.g. ABT-281, ASM981, etc.; corticosteroids; cyclophosphamide; azathioprine; methotrexate; leflunomide; mizoribine; mycophenolic acid; mycophenolate mofetil; 15-deoxyspergualine, Everolimus, FTY720 or an immunosuppressive homologue, analogue or derivative thereof; immunosuppressive monoclonal antibodies, e.g. monoclonal antibodies to leukocyte receptors, e.g. MHC, CD2, CD3, CD4, CD7, CD8, CD25, CD28, CD40. CD45, CD58, CD80, CD86 or their ligands; other immunomodulatory compounds, e.g. a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. LFA-1 antagonists, ICAM-1 or -3 antagonists, VCAM-4 antagonists or VLA-4 antagonists; or a chemotherapeutic agent.

Extracts from herbs, fungi, or other plants can also be used in combination with ACE. They include, without limitation, extracts from other Cordyceps species, including Cordyceps militaris, Cordyceps pruinosa, Cordyceps gunnii; extracts from rhubarb root, including Rhizoma rhei, Rheum palmatum, Daio, R. palmatum; extracts from Allium cepa, Allium savitum, Astragalus species; extracts from Mycelia sterilia (Fungus No. 10917) and/or compound WF10917, described in PCT International Publication No. WO 97/09298, which is incorporated herein by reference; extracts from Salvae miltiorrhizae; ST, a combination of 12 herbs (Kawachi, et al., Pathol Int, 44:339-44, 1994); prodigiosin or prodigiosin 25-C derived from Serratia marcescens (US Patent Publication No. 2005/0069560, which is incorporated herein by reference); extracts from Prunella vilgaris or molecules derived from the extract, including rosmarinic acid, or derivatives, including rosmarinyl isopropyl ester, rosmarinyl ethyl ester and/or (S)-rosmarinyl bis(tert-butyldimethylsilyl)ether (U.S. Pat. No. 6,140,363, which is herein incorporated by reference); Brazilian licorice extract, glycyrrhizin, periandrins and periandradulcins derived from licorice extract (U.S. Pat. No. 6,922,068, which in herein incorporated by reference), peptides isolated from the terrestrial bacterium Chromobacterium violaceum, including FR901228 and other compounds described in U.S. Pat. No. 7,041,639, which in incorporated herein by reference; and extracts or compounds from other fungi and plants.

### IV Immunosuppressants

ACE can be used in combination with 1, 2, 3 or more immunosuppressants. Examples of immunosuppressant agents include corticosteriods, calcineurin inhibitors, antiproliferative agents, SIP receptor agonists, kinase inhibitors, monoclonal antilymphocyte antibodies and polyclonal antilymphocyte antibodies.

431 Non-limiting examples of corticosteroids include Prednisone (Deltasone® and Orasone®) and Methylprednisolone (SoluMedrol®). Non-limiting examples of calcineurin inhibitors include Cyclosporine (Cyclosporin A, SangCya, Sandimmune®, Neoral®, Gengraf®), ISA, Tx247, ABT-281, ASM 981 and Tacrolimus (Prograf®, FK506). Non-limiting examples of antiproliferative agents include Mycophenolate Mofetil (CellCept®), Azathioprene (Imuran®), and Sirolimus (Rapamune®). Non-limiting examples of SIP receptor agonists include FTY 720 or analogues thereof. Non-limiting examples of kinase inhibitors include mTor kinase inhibitors, which are compounds, proteins or antibodies that target, decrease or inhibit the activity and/or function of members of the serine/threonine mTOR family. These include, without limitation, CCI-779, ABT578, SAR543, rapamycin and derivatives or analogs thereof, including 40-O-(2-hydroxyethyl)-rapamycin, rapalogs, including AP23573, AP23464, AP23675 and AP23841 from Ariad, Everolimus (CERTICAN, RAD001), biolimus 7, biolimus 9 and sirolimus (RAPAMUNE). Kinase inhibitors also include protein kinase C inhibitors, which include the compounds described the PCT publications WO 2005/097108 and WO 2005/068455, which are herein incorporated by reference in their entireties. Non-limiting examples of monoclonal antilymphocyte antibodies include Muromonab-CD3 (Orthoclone OKT3®), Interleukin-2 Receptor Antagonist (Basiliximab, Simulect®), and Daclizumab (Zenapax®). Non-limiting examples of polyclonal antilymphocyte antibodies include Antithymocyte globulin-equine (Atgam®) and Antithymocyte globulin-rabbit (RATG, Thymoglobulin®).

Immunosuppressant agents can be classified according to their specific molecular mode of action. The four main categories of immunosuppressant drugs currently used in treating patients with transplanted organs are the following. Calcineurin inhibitors inhibit T-cell activation, thus preventing T-cells from attacking the transplanted organ. Azathioprines disrupt the synthesis of DNA and RNA as well as the process of cell division. Monoclonal antibodies inhibit the binding of interleukin-2, which in turn slows down the production of T-cells in the patient's immune system. Corticosteroids suppress inflammation associated with transplant rejection.

Immunosuppressants can also be classified according to the specific organ that is transplanted. Basiliximab (Simulect) is also used in combination with such other drugs as cyclosporine and corticosteroids in kidney transplants. IL-2 blockers, including Simulect from Novartis, FK506 or CyA, MMF, prednisone or Rapamycin are also used in kidney transplants. Daclizumab (Zenapax) is also used in combination with such other drugs as cyclosporin and corticosteroids in kidney transplants. Similar drugs are used in heart transplants, but anti-lymphocyte globulin (ALG) is often used instead of Simulect. Muromonab CD3 (Orthoclone OKT3) is used along with cyclosporine in kidney, liver and heart transplants. Tacrolimus (Prograf) is used in liver and kidney transplants. It is under study for bone marrow, heart, pancreas, pancreatic island cell and small bowel transplantation.

Other immunosuppressants include, without limitation, SERP-1, a serine protease inhibitor produced by malignant rabbit fibroma virus (MRV) and myxoma virus (MYX), described in US Patent Publication No. 2004/0029801, which is incorporated herein by reference.

### V. Formulations, Administrations and Uses

ACE can be administered by any conventional route, in particular enterally, for example, orally, e.g. in the form of tablets or capsules, or parenterally, for example, in the form of injectable solutions or suspensions, topically, e.g. in the form of lotions, gels, ointments or creams, or in a nasal or a suppository form. The compositions of the present invention including ACE may be administered orally, parenterally, by inhalation spray, topically, rectally, nasally, buccally, vaginally or via an implanted reservoir. Pharmaceutical compositions comprising ACE in association with at least one pharmaceutical acceptable carrier or diluent can be manufactured in conventional manner by mixing with a pharmaceutically acceptable carrier or diluent.

The required dosage of ACE will of course vary depending on the mode of administration, the particular condition to be treated and the effect desired. In general, satisfactory results are indicated to be obtained orally at daily dosages of from about 10 to 1000 mg/kg per body weight, or about 50 mg/kg or lower. An indicated daily dosage in the larger mammal, e.g. humans, is in the range from about 100 mg/kg to about 1000 mg/kg, conveniently administered, for example, in divided doses up to four times a day or in retard form. Suitable unit dosage forms for oral administration comprise from ca. 1 to 50 mg active ingredient, or from ca. 50 to 500 mg active ingredient. When ACE is used in combination with other agents or immunosuppressants, it can be given simultaneously with the other drugs, or it can be given before the other drugs or sequentially.

As used herein, an effective amount is defined as the amount required to confer a therapeutic effect on the treated patient, and is typically determined based on age, surface area, weight and condition of the patient. The interrelationship of dosages for animals and humans (based on milligrams per meter squared of body surface) is described by Freireich et al., Cancer Chemother. Rep., 50: 219 (1966). Body surface area can be approximately determined from height and weight of the patient. See, e.g., Scientific Tables, Geigy Pharmaceuticals, Ardsley, New York, 537 (1970).

As used herein, the term “prevents” refers to avoiding the condition, so that the condition does not occur in any way. The term “inhibits” refers to a reduction in the condition, or a slowing of the progress of the condition. The term “reduces” refers to a lessening of the condition or a slowing of the progress of the condition.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “parenteral” as used herein includes subcutaneous, intravenous, intramuscular, intra-articular, intra-synovial, intrasternal, intraocular, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, the pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

The pharmaceutically acceptable compositions of this invention may also be administered topically, especially when the target of treatment includes areas or organs readily accessible by topical application, including diseases of the eye, the skin or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutically acceptable compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, the pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

The pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of the compounds of the present invention that may be combined with the carrier materials to produce a composition in a single dosage form will vary depending upon the host treated, the particular mode of administration. Preferably, the compositions should be formulated so that a dosage of between 0.01-100 mg/kg body weight/day of the modulator can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Depending upon the particular condition, or disease, to be treated or prevented, additional therapeutic agents, which are normally administered to treat or prevent that condition, may also be present in the compositions of this invention. As used herein, additional therapeutic agents that are normally administered to treat or prevent a particular disease, or condition, are known as “appropriate for the disease, or condition, being treated.”

In order that the invention described herein may be more fully understood, the following examples are set forth. It should be understood that these examples are for illustrative purposes only and are not to be construed as limiting this invention in any manner.

### EXAMPLES

### Example 1

Cordyceps sinensis Extract Preparation

Dried mycelium was obtained from Yantai Ruidong Science and Technology Developing Co., Ltd. (China). Extraction began by first grinding 7 g of dried mycelium to a fine powder. The powder was added to 100 mL of sterile, double-distilled water and boiled for 1 hour. Once cooled, the mixture was homogenized using a Polytron® tissue homogenizer until a consistent suspension was obtained. The homogenate was then centrifuged at 200×g to remove coarse particulates. The supernatant was further centrifuged at 15,000×g for 10 min at 4° C. to remove fine particulates and then freeze-dried and weighed. This extract, referred to as “Aqueous Cordyceps Extract” (ACE), was reconstituted at a known concentration in supplemented RPMI cell culture medium and stored at −70° C. until use. Samples were filter-sterilized using a 0.22 μm filter immediately prior to use.

### Example 2

Reduction of T-Cell Activation

Upon activation, immune cells, including T-cells, produce the cytokine IFN-γ. The effect of Cordyceps on T-cell activation was tested in vitro by examining IFN-γ production using a mixed lymphocyte reaction (MLR).

Male BALB/c, C57BL/6, C3H/HeJ, and C3H/HeOuJ mice were purchased from Jackson Laboratories (Bar Harbor, Me.). All mice were used at 6-8 wk of age and maintained in compliance with the Canadian Council on Animal Care guidelines. Food and water were provided ad libitum.

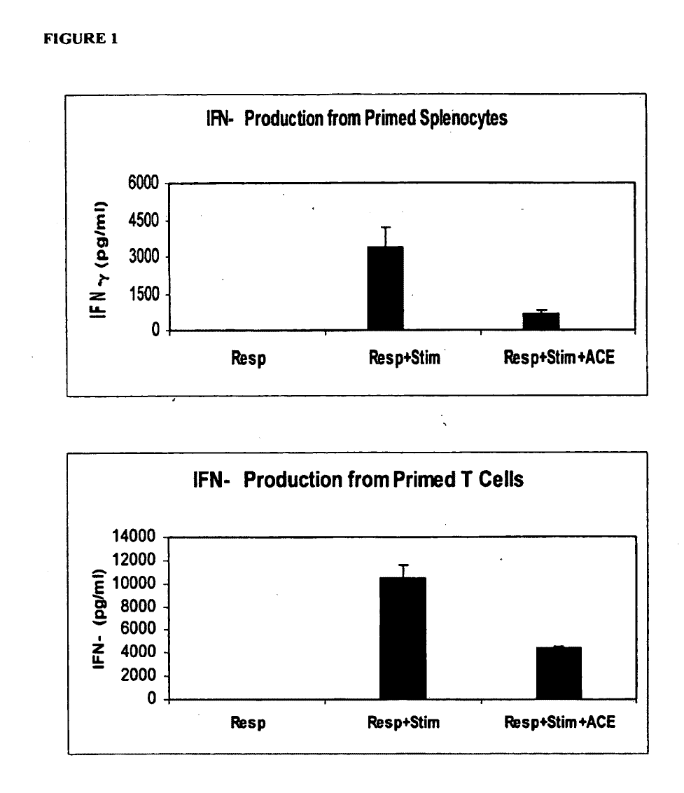

C57BL/6 mice were primed with allogeneic C3H/HeJ splenocytes. 7 days later splenocytes or purified T-cells were isolated from the primed C57BL/6 animals. The primed BL/6 cells (responders) were treated with an aqueous Cordyceps extract (ACE) for 24 h in the presence of C3H stimulators. IFN-γ levels were measured using ELISA. Results are shown in FIG. 1. “Resp” indicates untreated primed BL/6 splenocytes or purified T-cells (responders). “Resp+Stim” indicates primed BU6 splenocytes or purified T-cells (responders) treated with C3H stimulators. “Resp+Stim+ACE” indicates primed BL/6 splenocytes or purified T-cells (responders) treated with C3H stimulators and ACE. The data in FIG. 1 indicate that ACE reduces T-cell activation in both whole splenocyte populations and T-cell populations. The p values for IFN-γ production from primed splenocytes and T-cells are 0.004 and 0.0005, respectively.

Statistical analyses for the above and following examples were performed using Minitab® software (Minitab® Inc.; PA, USA) or GraphPad Prism® (GraphPad software; San Diego Calif., USA). Results were analyzed by the two-sample t test, ANOVA with post-hoc tests if significant, or the Mann-Whitney test for non-parametric data. p values less than 0.05 were considered to be significant.

### Example 3

Effect of ACE on IL-6 Production

IL-6 is a pleotropic cytokine and one of its effects is to enhance activation induced cell death of T cells. The early demise of populations of activated T cells leads to immunosuppression. Because macrophages produce IL-6, the effect of ACE on IL-6 production was examined.

Macrophages were isolated and cultured as follows. Mice were injected intraperitoneally (i.p.) with 2.5 mL of 4% (w/v) Brewer's thioglycollate (Sigma®; St. Louis, Mo.) to induce a peritoneal exudate. Four days post-injection cells were obtained by peritoneal lavage using 5 mL of cold cRPMI (ICN Biomedicals, Irvine; Calif.). cRPMI refers to RPMI supplemented with 10% fetal bovine serum (FBS), 100 U/ml penicillin, 100 μg/ml streptomycin, 50 mM β-mercaptoethanol, 2 mM L-glutamine, and 20 mM HEPES (Gibco BRL; Burlington, ON) and adjusted to pH 7.4. Cells obtained from the lavage were washed, counted, and cultured with cRPMI in 96-well flat-bottomed plates (Nunclon). Cells were plated at 2×10⁵ cells/well and incubated for 3 hours at 37° C. to allow for adherence. After incubation, cells were washed twice to remove non-adherent cells and 100 μL of fresh cRPMI was added to each well. Treatment agents were applied to the adherent macrophages diluted in 100 μL of media. LPS (E. coli serotype 055:55, Sigma) was used at a concentration of 10 ηg/mL. ACE was used at various concentrations. All wells had a final volume of 200 μL, and treatments were performed in triplicate. Macrophages were cultured at 37° C. and supernatants collected after 48 hours for analysis.

In some experiments, the murine macrophage cell line J774 was used. J774 cells were obtained from ATCC (Virginia, US), maintained in cRPMI (as above) at 37° C., and plated at 3.5×10⁴ cells/well.

Supernatants from the cell cultures were analyzed for IL-6 by a sandwich ELISA according to manufacturer's instructions (BD Biosciences). Briefly, flat-bottomed 96-well ELISA plates were coated with anti-mouse IL-6 antibody in carbonate buffer and incubated overnight at 4° C. After washing the plates and subsequent blocking, standards or test supernatants were applied to wells and incubated at room temperature for 2 hours. After washing well, plates were incubated with a biotinylated detection antibody and an avidin-horseradish peroxidase conjugate for 1 hour at room temperature. Samples were developed using TMB substrate solution and the reaction stopped with 1 M phosphoric acid. ELISA plates were read using a plate spectrophotometer (Molecular Devices, California) at 450 ηm. Results were analysed using SOFTMax® Pro software (Molecular Devices).

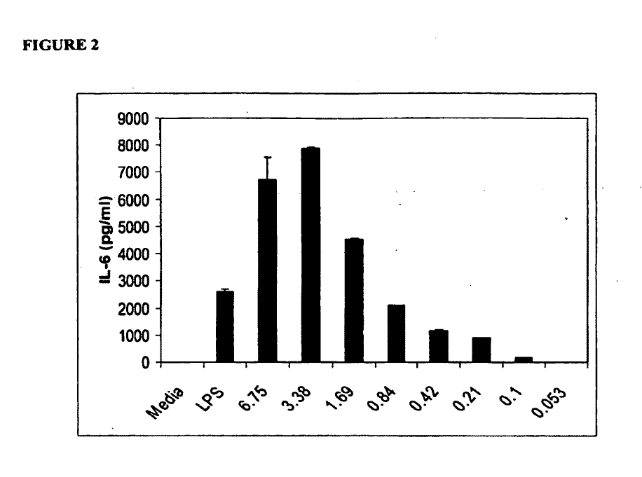

Dose-dependent production of IL-6 was observed after treatment with ACE, as shown in FIG. 2. Means and standard deviations of triplicate samples are shown. Data shown are representative of three separate experiments. ACE was tested at dilutions from 6.75 mg/mL to 0.053 mg/mL. ACE induced IL-6 at a variety of doses and was more effective than the positive control (10 ηg/mL LPS) at the higher doses (IL-6, p=0.0016).

### Example 4

Fractionation of ACE

Aqueous Cordyceps sinensis extract was fractionated using PALL Life Sciences Microsep Concentration filters (Ann Arbor, Mich.) with a molecular weight cut-off of 300 kDa and Millipore Centricon® (Bedford, Mass.) centrifugal filter devices with a molecular weight cut-off of 100 kDa. For the >300 kDa and <300 kDa fractions, the Pall concentration filters were washed twice with 3 mL of sterile, distilled water at 2500×g for 10 minutes. ACE was then added to the washed filter and centrifuged at 2500×g for 45 minutes. The top layer remaining in the filter, containing the >300 kDa fraction, was removed and reconstituted to the original volume using sterile, distilled water. Both fractions were stored at −20° C. until use, or the <300 kDa fraction was used to further separate the extract into a >100 kDa fraction and a <100 kDa fraction. To do this, the Millipore Centricon® filters were washed with 2 mL of sterile, distilled water at 1000×g for 10 minutes. The filter was removed, inverted, and centrifuged for an additional 5 minutes. This washing protocol was performed twice. ACE <300 kDa was then added to the washed filter and centrifuged at 1000×g for 5-10 min; enough to allow some liquid to remain in the top layer. The top layer (the >100 kDa fraction) was reconstituted to its original volume using sterile, distilled water. Both fractions were stored at −20° C. until use. All fractions were filter sterilized using a 0.22 μm syringe filter before use.

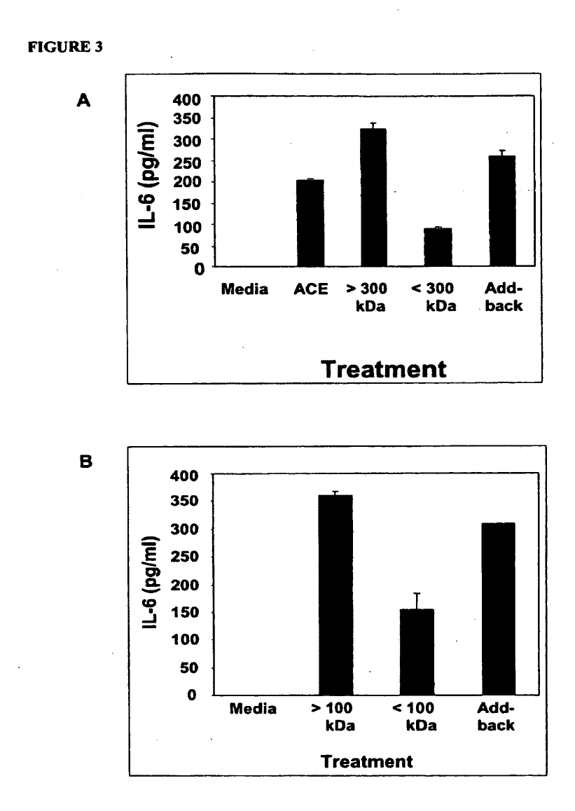

The fractions were tested for the production of IL-6 in macrophages. FIG. 3 shows that the larger molecular weight fractions, >300 kDa and >100 kDa elicited higher IL-6 production from macrophages, but that the smaller molecular weight fractions of <300 kDa and <100 kDa also elicited IL-6 production.

### Example 5

Acute Rejection

Acute rejection follows cardiac transplantation. There has been considerable success in reducing this rejection by the use of calcineurin inhibitors such as cyclosporine A (CyA). However, side effects such as nephrotoxicity, increased incidence of opportunistic infection, and increased incidence of neoplasms have limited the benefit of these therapies. Use of CyA at a lower dose, in conjunction with another therapy may prove more acceptable. We tested the ability of ACE to act as an adjunct therapy for acute rejection using a rat heterotopic heart transplant model. Rats were fed 100 mg/kg of ACE on days −1, 0, 1 and every second day following. Hearts from Brown-Norway rats were transplanted into Lewis rats. Fully disparate heart grafts were transplanted. CyA was injected subcutaneously every day post-transplant at a sub-therapeutic dose of 2-10 mg/kg or a therapeutic dose of 50 mg/kg. Animals were sacrificed at day 12±1 day and histological sections of heart tissue were made.

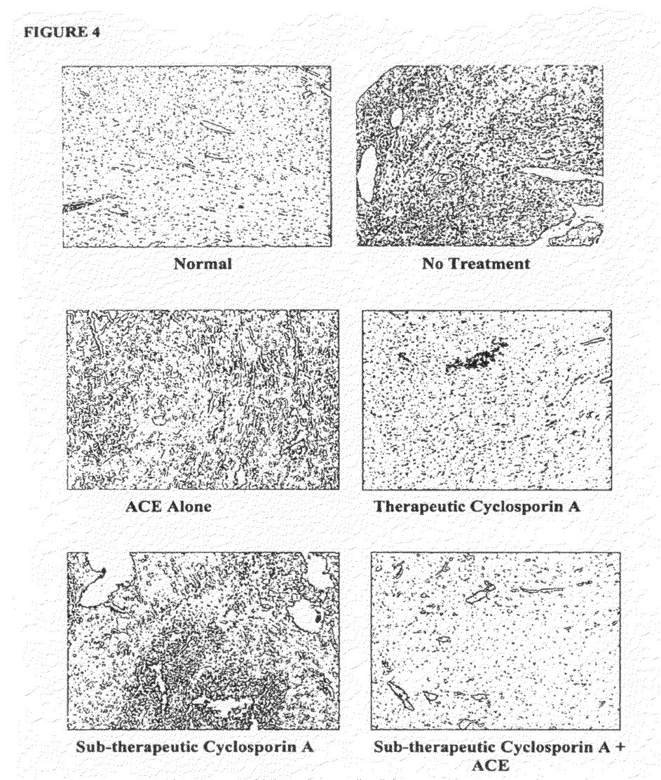

FIG. 4 depicts photomicrographs of haematoxylin and eosin (H&E) stains of heart tissue at 10× from the rats. These results show that ACE alone improves transplanted heart architecture somewhat over normal, and in combination with low dose CyA improves heart architecture similar to that of therapeutic CyA after transplantation.

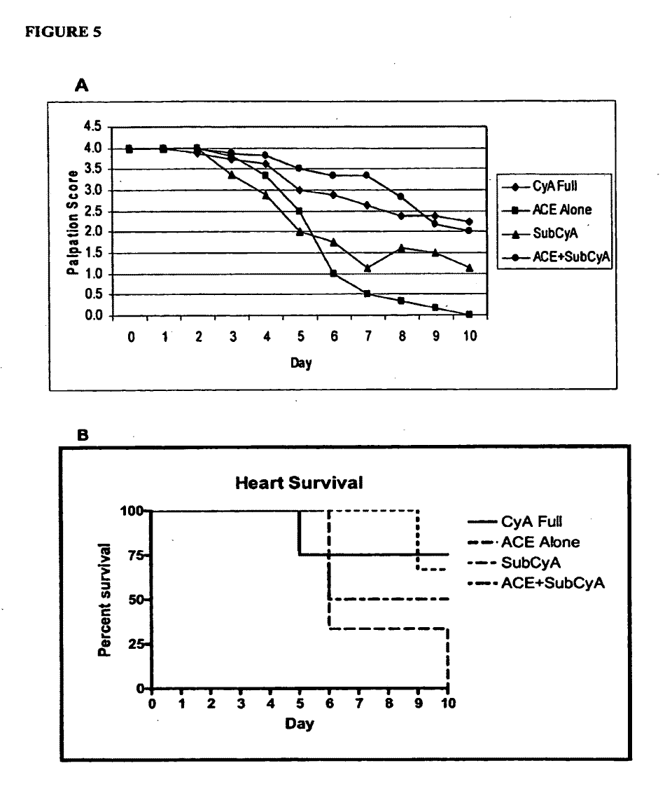

Transplant function was evaluated by regular palpation graded on a scale 4 (functioning well) to 0 (not beating); hearts were considered acutely rejected when palpation scores were less than 1. FIG. 5A shows palpation scores of transplanted hearts in rats treated with CyA alone, ACE alone or a combination. The results indicate that ACE in combination with a subtherapeutic dose of CyA has a similar effect as a therapeutic dose of CyA. FIG. 5B shows a Kaplan-Meier plot of the results, which also indicate that the combination of ACE and a subtherapeutic dose of CyA has a similar effect on heart survival as a therapeutic dose of CyA. These results suggest that ACE acts synergistically with low-dose CyA to reduce myocyte destruction mediated by acute rejection.

### Example 6

Chronic Rejection

The leading cause of long-term graft failure after cardiac transplantation is allograft vasculopathy (AV). In AV, a neointimal lesion (NI) forms in the lumen, occluding the vessel, resulting in loss of viability of the transplanted organ. Currently, there is no treatment available to prevent AV. The ability of ACE to reduce neointimal formation in a mouse and rat abdominal aorta transplant models was examined. These are reliable animal models for the study of allograft vasculopathy in cardiac transplantation.

A. Animals

Six to eight wk old C3H/HeJ mice (Jackson Laboratories) were used as the donors and age-matched C57BL/6 mice (Jackson) as the recipients. These strains are chosen because they are fully disparate and, therefore, provide an allogeneic environment for the graft. For transplants in rats, Brown-Norway rats were donors and Lewis rats were recipients. Animals were maintained in compliance with the Canadian Council on Animal Care guidelines and are fed food and water ad libitum.

B. Transplants

Donors (C3H/HeJ) were anaesthetized with 55 mg/kg of sodium pentobarbitol, i.p. prior to surgery. Once anaesthetized, the abdominal area was shaved and cleaned using an alcohol/Betadine solution. A midline incision was made in the abdomen using a No. 11 scalpel blade. Next, a 5-0 suture was used to pull back the skin and muscle to expose the abdominal area. The intestines were moved to the left and kept moist with wet gauze. The abdominal aorta was separated from the inferior vena cava using a No. 5 forceps. Two microvascular clamps (micro serrifines) were inserted to obtain proximal and distal control of the donor aorta. An abdominal aortic segment was harvested and flushed with saline using a 21 gauge needle for rats and a 26 gauge needle for mice. The aortic segment was placed in cold saline while waiting for transplant.

Recipients (C57BL/6 mice) were anaesthetized with 55 mg/kg of sodium pentobarbitol, i.p. prior to surgery. Once anaesthetized, the abdominal area was shaved and cleaned using an alcohol/Betadine solution. A midline incision in the abdomen was made using a No. 11 scalpel blade, and the intestines were moved aside and kept moist with wet gauze. The abdominal aorta was separated from the inferior vena cava and surrounding tissues. The aorta was clamped with two microvascular clamps to obtain proximal and distal control of the recipient aorta. A single cut was made with microscissors in the recipient abdominal aorta to allow for placement of the donor aorta. The donor segment was placed into the abdominal space of the recipient aorta targeted for transplant. Using end to end microsurgical anastomotic technique, the donor segment was sutured to the recipient aorta using single interrupted sutures (11-0 nylon sutures). The clamps were removed and blood flow was re-established. The intestine was gently repositioned into the abdomen. The muscle was closed first, then the skin with a continuous stitch using a 5-0 or 4-0 suture (the skin and the muscle can be sutured together in mice). The recipient was placed on a heating pad overnight for recovery. The transplanted animal was monitored and treated for the eight weeks of the study.

C. Cordyceps sinensis Feeding Protocol

One day prior to surgery (day minus one), the recipient animal is fed an aqueous extract of Cordyceps sinensis (ACE) in a volume of 0.1 mL using an infant feeding tube attached to a 1 mL syringe. On the day of the transplant (day 0), this feeding procedure is repeated prior to surgery. Once the animal has been successfully transplanted and recovered overnight (day 1), it is once again fed with the extract. The daily dose is 50 mg/kg. The feeding continues every second day following for 8 weeks.

D. Cyclosporine Injection Protocol

One day following transplantation (day 1), the animal is injected sub-cutaneously with the immunosuppressant Cyclosporin A (Sandimmune-γ). Every day following, for 8 weeks, the animal is injected with CyA in this manner.

E. Tissue Processing

At eight weeks post-transplantation, the animal is anaesthetized with sodium pentobarbital, and the graft is removed (the animal is euthanized at this point). The graft is then fixed in 10% formalin overnight, washed in phosphate buffer, placed in 70% ethanol. The tissue is embedded in paraffin blocks and 5 μm sections are cut (using a microtome) in preparation for staining procedures. In some instances it may be necessary to use frozen tissue rather than paraffin-embedded. In such cases, the harvested graft is immersed in a cryopreservative and flash frozen using liquid nitrogen. The tissue preserved in this manner is then cut using a cryotome in preparation for staining procedures.

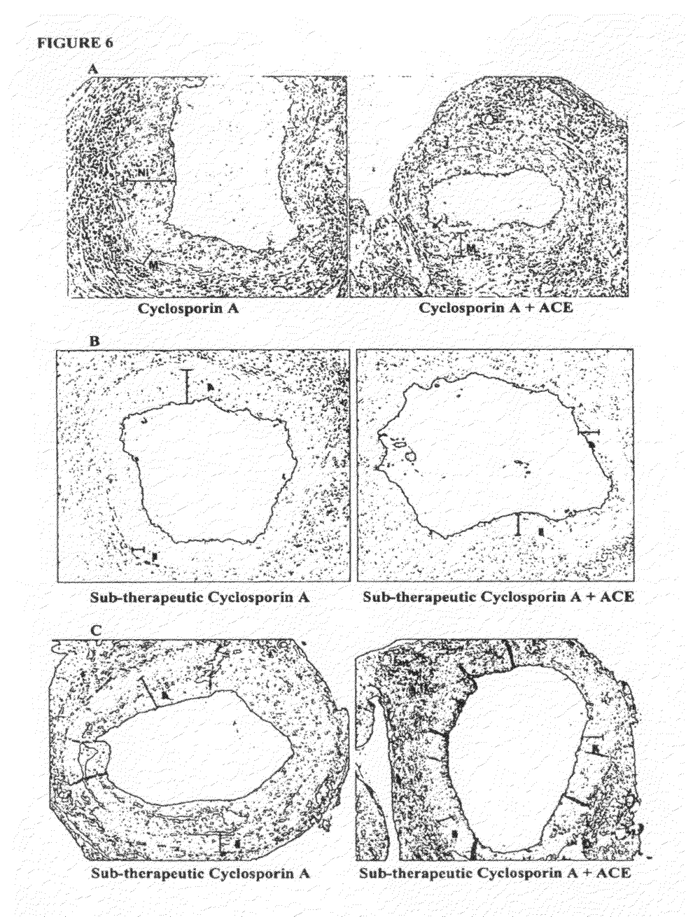

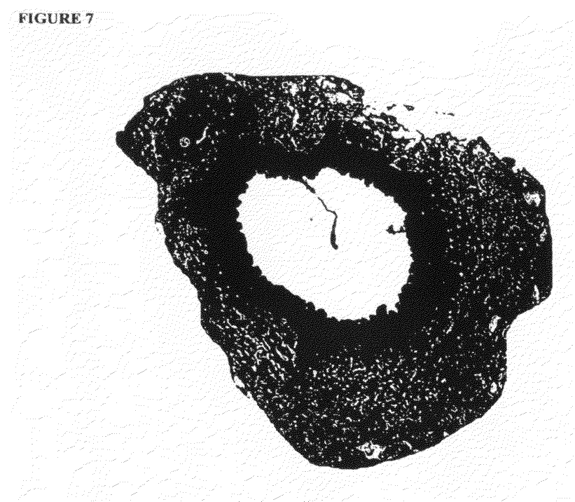

CyA was injected subcutaneously at a therapeutic dose post-transplant to ablate acute rejection such that only chronic rejection is measured in this model. The results in FIG. 6 show photomicrographs of H&E stains at 20× of the vasculature of transplanted aortae and show that ACE along with CyA either ablates (FIG. 6A) or decreases (FIG. 6B, C) the formation of a neointima, suggesting that ACE with CyA prevents allograft vasculopathy. FIGS. 6A and 6B are photomicrographs of transplanted aortae from mice, and FIG. 6C are photomicrographs of transplanted aortae from rats. FIG. 7 shows a photomicrograph of a transplanted aorta from a mouse that was treated with the fraction of ACE containing molecules of less than 100 kDa. The results, which show a decrease in neointimal formation, indicate that the immunosuppressant activity of ACE resides, at least in part, in the smaller molecular weight fraction.

### Example 7

Modulation of Gene Expression

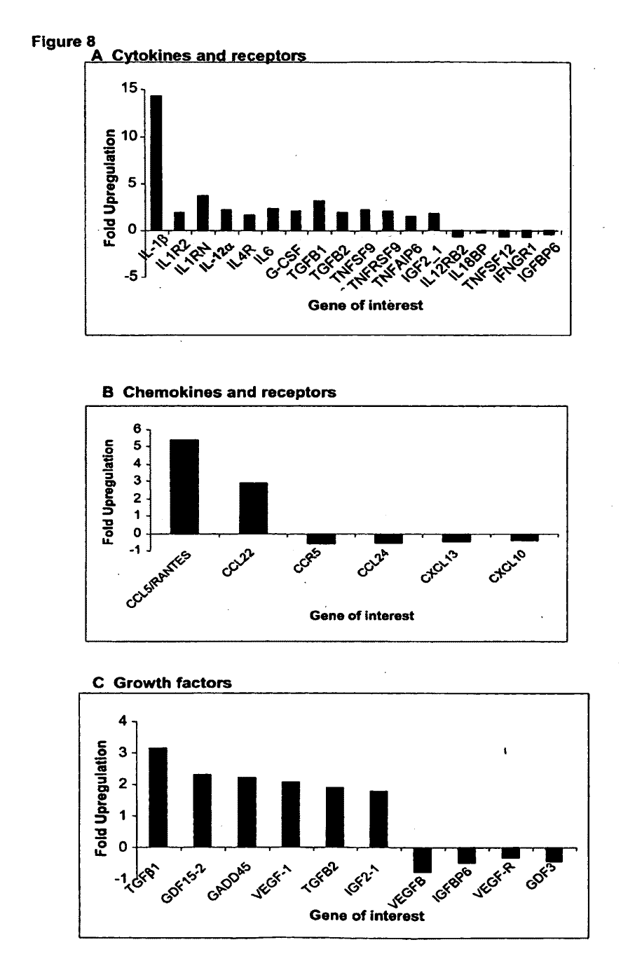

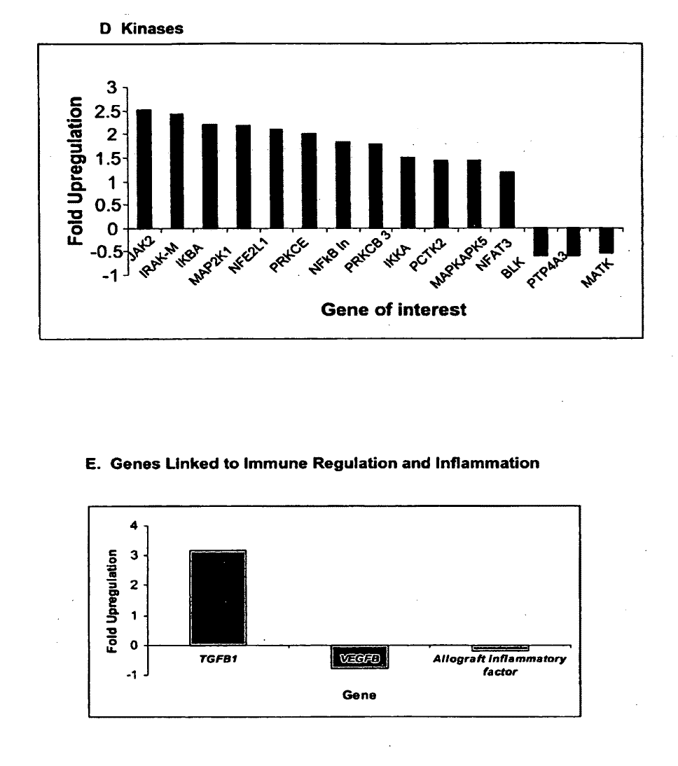

Gene expression can provide information as to how ACE is having its immunosuppressive effect. Gene activity for 1076 immunological genes was examined using microarray analysis of ACE-treated peritoneal elicited murine macrophages for 18 hours. mRNA was isolated from untreated macrophages and macrophages treated with ACE. Fluorescent labeled cDNAs generated from the mRNAs were hybridized to the arrays. The results indicated genes upregulated or downregulated by ACE. FIG. 8 shows the activity of a sample of genes linked to immune regulation and inflammation in transplants. Upregulated genes include the cytokines and receptors IL-1β, IL-6, and TNFSF; chemokines and receptors CCL5/RANTES and CCL22; growth factors TGFβ1, GDF15-2, GADD45 VEGF1, TGFβ2 IGF2-1 and VEGF2; and kinases JAK2, IκB4, MAPK1 and NFκB. In addition, allograft inflammatory factor is down regulated. These results indicate that ACE can upregulate immunosuppressive genes, such as TGFβ while downregulating genes involved in inflammation, fibrosis and neointimal lesion formation such as VEGF and allograft inflammatory factor.

### Example 8

Effect of ACE on Macrophages

Dose-dependent production of IL-6 from macrophages was observed after treatment with ACE. (See Example 3.) Macrophage activation is a major component of innate immunity. Resting macrophages can become activated through receptor-mediated responses initiated by a large variety of stimuli, including cytokines, bacterial cell wall components such as lipopolysaccharide (LPS) or fungal polysaccharides. Once activated, macrophages produce cytokines such as IL-6 and tumor necrosis factor (TNF-α).

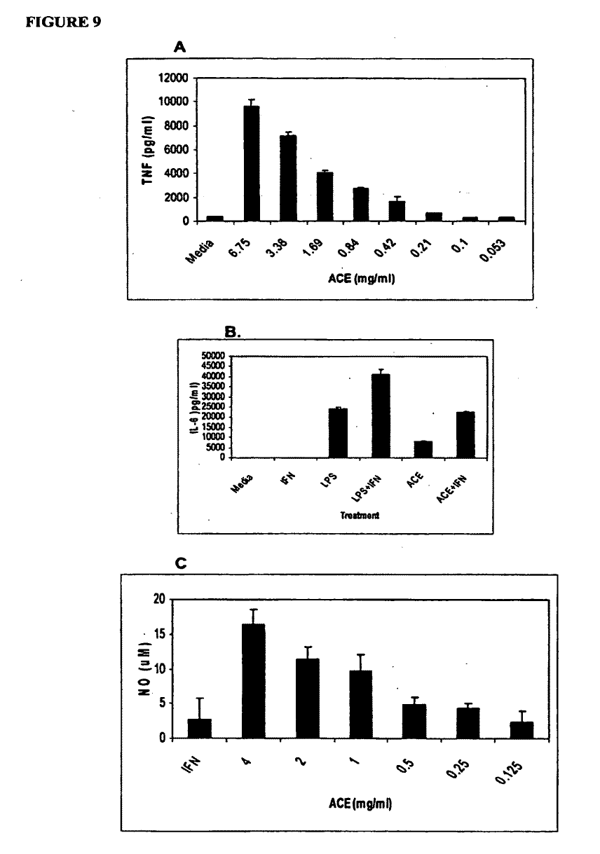

The effect of ACE on the production of TNF-α was examined. Macrophages were isolated, cultured and treated as in Example 3. TNF-α levels were detected using a sandwich ELISA, as for IL-6 in Example 3. Results in FIG. 9A show that ACE induces TNF production at a variety of doses. (p=0.0016.) Means and standard deviations of triplicate samples are shown. Data shown are representative of three separate experiments.

Co-administration of IFN-γ with strong macrophage triggers generally has a synergistic effect on cytokine production. Thus, the effect between ACE and IFN-γ was determined in macrophages. Macrophages were isolated and cultured as described above. Elicited murine peritoneal macrophages were cultured for 48 hours with ACE or LPS in the presence or absence of IFN-γ. The final concentration of recombinant mouse IFN-γ (Pierce Endogen) used was 2 U/ml. The results, shown in FIG. 9B, demonstrate that, for the positive LPS control (p=0.0067 and for ACE (p<0.0001) co-administration of IFN-γ results in a synergistic increase in IL-6 production. Means and standard deviations of triplicate samples are shown. Data shown are representative of three separate experiments.

In addition to cytokine production, macrophage activation leads to the production of reactive nitrogen intermediates, such as nitric oxide (NO). Accordingly, the level of NO produced by macrophages cultured with ACE was examined. After 48 hours, samples (50 μL) from culture supernatants were transferred into individual wells of a 96-well flat-bottomed plate (in triplicate). Wells were subsequently treated with 25 μL each of 1% sulfanilamide (Griess R1) and then 0.1% N-(1-napthyl) ethylene-diamene (Griess R2; Cedarland Laboratories) and left to incubate for 10 min at room temperature. The plate was read at 550 ηm using a plate spectrophotometer. Doubling dilutions of sodium nitrite were used as standards. Results were analyzed using SOFTMax® Pro software.

The results in FIG. 9C show that ACE, in the presence of IFN-γ, induces NO production by macrophages dependent manner.

### Example 9

Pattern Recognition Receptors

Although the elements of the innate immune response, such as macrophages, do not have the ability to recognize specific antigens, it has long been postulated that a degree of recognition of pathogens has evolved over time. Recently, evidence has accumulated that innate cells, such as macrophages, possess receptors able to recognize patterns associated with surface molecules on pathogens. These patterns are often called pathogen-associated molecular patterns (PAMP).

Toll-like receptors (TLRs) are leucine-rich, highly conserved proteins that bind to the PAMP and activate macrophages and other innate immune cells. TLRs also help initiate adaptive immunity by releasing cytokines. LPS activation of macrophages involves engagement of TLR4 (Poltorak, et al., Science, 282:2085-88, 1998). Because the activation profile of LPS and ACE are similar, TLR4 as a potential mechanism of action for ACE was examined.

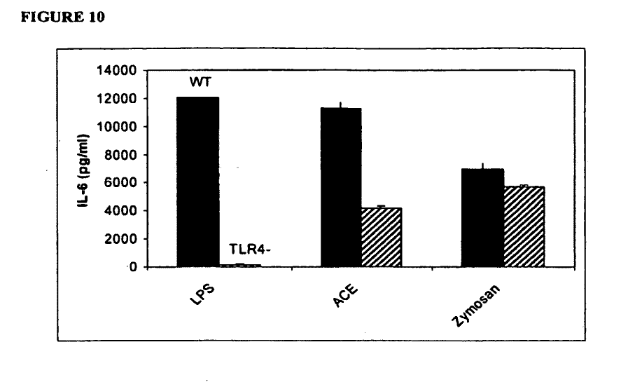

Macrophages, which produce IL-6, recognize motifs associated with pathogens through Toll-like receptor 4 (TLR4) and Toll-like receptor 2 (TLR2), two of the most well-studied cell-surface pattern recognition receptors. We assessed the role of TLR4 in ACE activation by using elicited macrophages from mice with defective TLR4 (C3H/HeJ). As a control we used macrophages from a wild-type (age and sex-matched) strain of mice, known to possess functional TLR4 (C3H/HeOuJ). Elicited peritoneal macrophages from TLR4-deficient (C3H/HeJ) or strain-specific wild type mice (C3H/HeOuJ) were cultured for 48 hours with 10 ηg/mL LPS or 3.4 mg/mL ACE. Zymosan, an insoluble carbohydrate from yeast cell walls, was used as a positive control at a concentration of 0.01%. Zymosan induces cytokine production by macrophages through TLRs. Supernatants were assayed for IL-6 using ELISA. Results in FIG. 10 show means and standard deviations of triplicate samples. Data are representative of three separate experiments.

Both LPS and ACE stimulated a robust IL-6 response in the wild type mice. In contrast, in the TLR4 defective mice, IL-6 production in response to LPS stimulation was completely ablated (FIG. 10). A significant (p=0.0013) but not complete reduction in IL-6 production in response to ACE was seen in the TLR4 defective mice. This implicates TLR4 in ACE-mediated macrophage activation, but clearly indicates that other mechanisms are involved.

### Example 10

Effect of ACE on MAPKinase

Activation of adaptor molecules by TLR normally leads to activation of the mitogen activated protein kinase (MAPK) intracellular signaling pathways. These pathways involve a cascade of signaling from cell-surface receptors to transcription factors and ultimately the regulation of specific gene expression. LPS activates murine macrophages by increasing tyrosine phosphorylation of a variety of intracellular proteins, including MAP kinases. Of these, the isoforms, ERK1 and ERK2 are the most well recognized. In addition, there is evidence showing a role for the p38 MAP kinase in regulating cytokine expression and thus, in activating the immune response after LPS stimulation (Han, et al., Science, 265:808-11, 1994).

Because activation of TLRs most often results in the activation of MAPK pathways, we assessed whether ACE would induce the phosphorylation and activation of ERK or p38, two important contributors to the MAPK pathway. Specific inhibitors of these protein kinases were first used.

Adherent macrophages were pre-incubated with media, vehicle (0.06% DMSO; Sigma), 25 μM ERK inhibitor, (PD98059; Calbiochem), or 25 μM p38 inhibitor (SB203580; Calbiochem) for 30 min at 37° C. before being treated with test agent. The cells were further incubated for 48 hours with ACE (3.4 mg/mL) or Zymosan (0.01%). DMSO was used as a vehicle control. IL-6 production was assayed using ELISA.

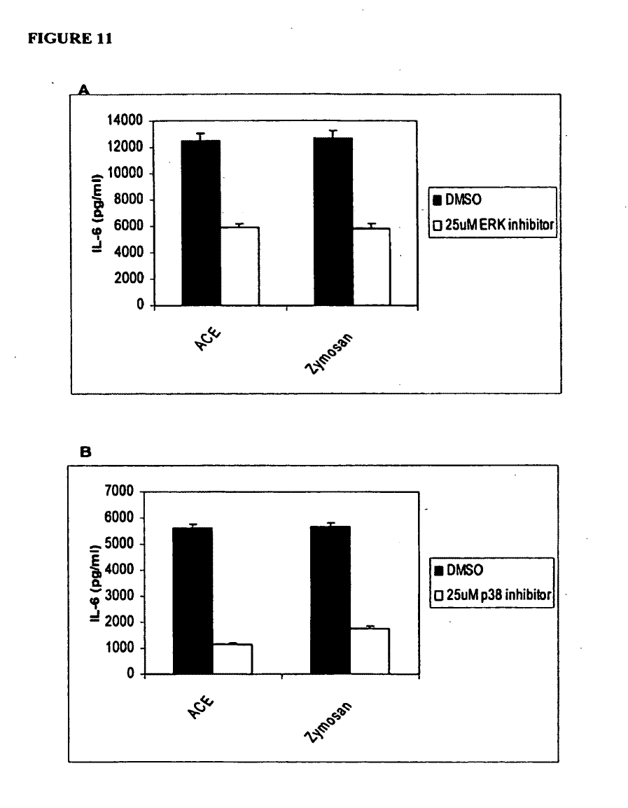

Results in FIG. 11 demonstrate that the ERK inhibitor PD98059 and the p38 inhibitor SB203580 both caused marked decreases of IL-6 production by the ACE treated macrophages in comparison to cultures which did not contain the inhibitor (p=0.0025 and p=0.0006, respectively). Zymosan, which is known to use ERK and p38 during activation was used as a control for the inhibitors (inhibition p<0.05).

Phosphorylation of ERK and p38 was confirmed using Western Blotting with antibodies specific for phosphorylated ERK and p38.

Cell lysates were prepared as follows. Fresh media was added to cultured peritoneal BALB/c macrophages 2 hours before treating cells for indicated time points with 3.4 mg/ml of ACE. Cells were washed once with sterile saline and transferred to lysis buffer (1% Triton-X 100, 150 mM NaCl, 10 mM Tris HCl, 1 mM EDTA, 1 mM EGTA, 0.2 mM sodium ortho-vandate, Nonidet P-40, and protease inhibitors cocktail tablets (Boehringer Mannheim, Cat.# 1873580) and incubated for 40 min on ice.

Western blots were prepared as follows. Cell lysates were diluted 1:3 in sample buffer (187.5 mM Tris-HCl, 30% glycerol, 6% SDS, 15% β-mercaptoethanol, 0.075% Bromophenol blue) and boiled for 5 min before loading onto 12% SDS-polyacrylamide electrophoresis gels. Gels were run at constant voltage (200 V) for 40 min. Proteins were transferred to nitrocellulose at 400 mA for 1 hour in buffer containing 25 mM Tris base, 192 mM glycine, and 20% methanol. After transfer, nitrocellulose was washed with 0.05% Tween-20 in Tris buffered saline (TBST) and blocked for 1 hour at room temperature in TBST containing 10% (w/v) skim milk powder. Membranes were washed and incubated with p44/p42 MAP kinase (ERK antibody) or p38 MAP kinase antibody (Cell Signaling Technology) at a dilution of 1:1000 in TBST with 5% BSA overnight at 4° C. The membrane was then washed, incubated with 1:2000 donkey anti-rabbit IgG HRP-conjugated antibody (Santa Cruz Biotechnology). Specific bands were detected using Enhanced Chemi-Luminescence (Pierce). Ponceau S staining of the nitrocellulose was then carried out to ensure samples were loaded evenly.

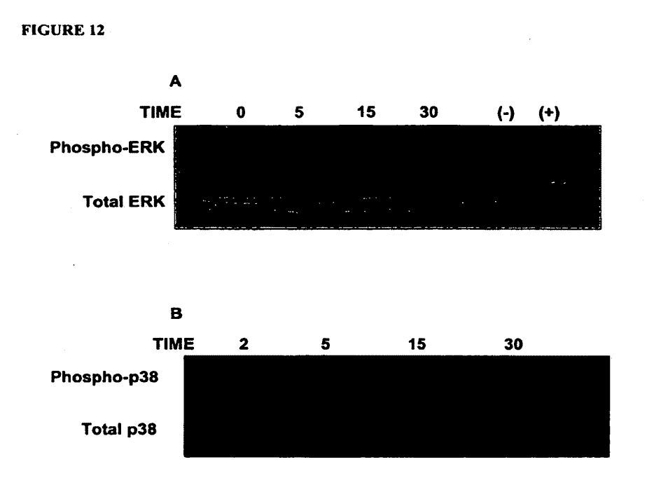

FIG. 12 shows that ACE induces the phosphorylation of these proteins, implicating the MAPK pathway is ACE-mediated macrophage activation.

### Example 11

Test of ACE for Endotoxin Activity

Many of the effects seen with ACE mirror the known effects of bacterial endotoxin, including LPS. Therefore, it was necessary to confirm that results were not influenced by the presence of bacterial endotoxin in our samples. Polymyxin B (PMB) is an inhibitor of LPS, which functions by interacting with bacterial phospholipids. As an antibiotic, this property of PMB leads to the eventual destruction of the cell walls of Gram-negative bacteria.

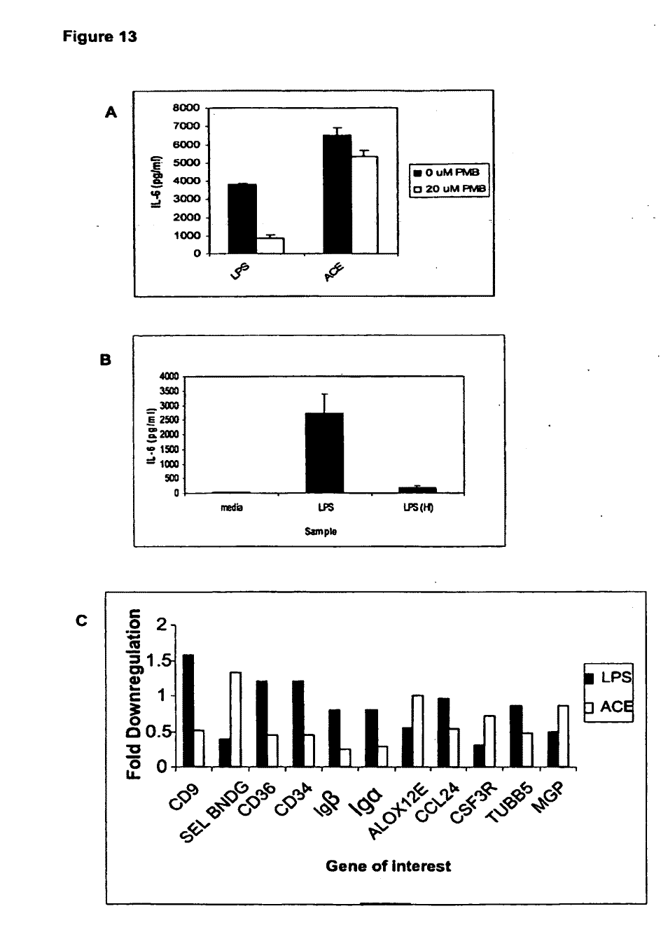

To test for LPS contamination, macrophages were pre-treated with Polymyxin B, followed by incubation with ACE. Peritoneal elicited macrophages were pre-incubated with media or LPS inhibitor Polymyxin B (Calbiochem) at 20 μg/ml for 30 min at 37° C. Cells were further incubated with LPS (10 ηg/ml) or ACE (3.4 mg/ml). The results in FIG. 13A show that Polymyxin B markedly reduced LPS activity (almost 80%) but not the activity of ACE. Results are shown as levels of IL-6 production, which were measured using ELISA. Means and standard deviations of triplicate samples are shown. Data are representative of three separate experiments.

It has been reported that boiling LPS for one hour will significantly reduce its activity. To test the effect of heat on LPS, peritoneal elicited macrophages were incubated with LPS (10 ηg/mL) or heat-inactivated LPS (HI) (10 ηg/mL). Results in FIG. 13B demonstrate that boiling does reduce LPS activity significantly (p=0.003). Means and standard deviations of triplicate samples are shown. Data are representative of three separate experiments. Because boiling is used to prepare ACE, any endotoxin activity would have been eliminated during extract preparation.

To further confirm that ACE does not contain endotoxin activity, microarray data obtained from treating macrophages with LPS was compared with that obtained with ACE. FIG. 13C shows that the levels of expression of various genes is different in macrophages treated with LPS compared with those treated with ACE.

Taken together, these data confirm that the observed activation of macrophages was due to stimulation by components of the C. sinensis extract and not by bacterial endotoxin.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages and modifications are within the scope of the following claims. All references cited herein are incorporated herein in their entirety.

What is claimed is:

1. A method for preventing or treating chronic rejection following organ tissue or cell transplantation, comprising administering to an animal an effective amount of an aqueous extract of Cordyceps sinensis.

2. The method of claim 1, wherein the transplanted organ, tissue or cells are selected from kidney, heart, heart valve, arteries, vessels, liver, lung, hemopoetic stem cells, pancreas, cornea, small bowel, and skin.

3. The method of claim 1, wherein the transplanted organ, tissues or cells include kidney and heart.

4. The method of any of claims 1-3, wherein the aqueous extract of Cordyceps sinensis consists of molecules of 500 kDa or less.

5. The method of any of claims 1-3, wherein the aqueous extract of Cordyceps sinensis consists of molecules of 300 kDa or less.

6. The method of any of claims 1-3, wherein the aqueous extract of Cordyceps sinensis consists of molecules of 200 kDa or less.

7. The method of any of claims 1-3, wherein the aqueous extract of Cordyceps sinensis consists of molecules of 100 kDa or less.

8. The method of any of claims 1-3, wherein the aqueous extract of Cordyceps sinensis consists of molecules of 50 kDa or less.

9. The method of claim 3, wherein the aqueous extract of Cordyceps sinensis prevents, inhibits or reduces allograft vasculopathy.

10. The method of claim 3, wherein the aqueous extract of Cordyceps sinensis prevents, inhibits or reduces neointimal formation in arteries or vessels.

11. The method of claim 1, further comprising administering an immunosuppressant.

12. The method of claim 11 or 12, wherein the immunosuppressant is selected from the group consisting of Cyclosporin A, FK506, other calcineurin inhibitors, Rapamycin, MMF, Azathioprine, FTY720, Everolimus, and kinase inhibitors.

13. The method of claim 11 or 12, further comprising administering one or more immunosuppressants.

14. A mixture comprising an aqueous extract of Cordyceps sinensis, wherein the extract consists of molecules of 500 kDa or less.

15. A mixture comprising an aqueous extract of Cordyceps sinensis, wherein the extract consists of molecules of 300 kDa or less.

16. A mixture comprising an aqueous extract of Cordyceps sinensis, wherein the extract consists of molecules of 200 kDa or less.

17. A mixture comprising an aqueous extract of Cordyceps sinensis, wherein the extract consists of molecules of 100 kDa or less.

18. A method of preparing an aqueous extract of Cordyceps sinensis comprising molecules of 300 kDa or less, wherein an aqueous extract of Cordyceps sinensis is passed through a filter.

19. A method of preparing an aqueous extract of Cordyceps sinensis comprising molecules of 100 kDa or less, wherein an aqueous extract of Cordyceps sinensis is passed through a filter.

20. A pharmaceutical composition comprising an aqueous extract of Cordyceps sinensis and a pharmaceutically acceptable carrier.

21. The pharmaceutical composition of claim 20, wherein the aqueous extract of Cordyceps sinensis comprises molecules whose molecular weight is 300 kDa or less.

22. The pharmaceutical composition of claim 20, wherein the aqueous extract of Cordyceps sinensis comprises molecules whose molecular weight is 100 kDa or less.

23. The pharmaceutical composition of any of claims 20-22, further comprising an immunosuppressant.

24. The composition of claim 23, wherein the immunosuppressant is selected from the group consisting of Cyclosporin A, FK506, other calcineurin inhibitors, Rapamycin, MMF, Azathioprine, FTY720, Everolimus, and kinase inhibitors.

25. The composition of claim 22 or 24, further comprising one or more additional immunosuppressants.

26. The composition of claim 24, wherein the immunosuppressant is Cyclosporin A.
